(19) United States
(12) Patent Application Publication
(10) Pub. No.: US 2013/0046011 A1
(43) Pub. Date: Feb. 21, 2013

(54) HOT-MELT EXTRUDED COMPOSITIONS CONTAINING PLANT-DERIVED PHENOLIC MATERIALS AND PROCESSES FOR THE PREPARATION THEREOF

(71) Applicant: Board of Governors for Higher Eduction, State of Rhode Island and Providence Plantations, Providence, RI (US)

(72) Inventors: Gowri Dorairaju, Marlborough, MA (US); Anthony E. Listro, Woodstock, MA (US); David R. Worthen, Wakefield, RI (US)

(73) Assignee: Board of Governors for Higher Education, State of Rhode Island and Providence Plantations, Providence, RI (US)

(21) Appl. No.: 13/661,268

(22) Filed: Oct. 26, 2012

Related U.S. Application Data
(63) Continuation of application No. 13/399,203, filed on Feb. 17, 2012.
Application No. 13/399,203 is a continuation of application No. PCT/US2010/046405, filed on Aug. 24, 2010.
(60) Provisional application No. 61/236,181, filed on Aug. 24, 2009.

(51) Int. Cl. A61K 31/05; A61K 31/353

(52) U.S. Cl. 514/456; 514/733

(57) ABSTRACT

A hot-melt extruded composition is disclosed that includes about 20-80% wt. of a plant-derived phenolic material; about 20-85% wt. of one or more edible or bioerodible excipients; about 0-40% wt. of a surface active material; about 0-40% wt. of an oral absorption enhancer; and about 0-10% wt. of one or more pharmaceutical or food grade additives. The composition has been hot-melt extruded at a temperature substantially below the melting point of the plant-derived phenolic material to produce a hot-melt extruded composition wherein substantial degradation of the plant-derived phenolic material has not occurred.

### PRIORITY

The present application is a continuation application of PCT/US2010/046405 filed on Aug. 24, 2010, which claims priority to U.S. Provisional Patent Application Ser. No. 61/236,181 filed Aug. 24, 2009, the disclosures of each of which is hereby incorporated in its entirety.

### BACKGROUND OF THE INVENTION

1. Field of the Invention

The invention generally relates to composition delivery systems and methods in pharmaceutical applications, and relates in particular to compositions for improving the stability, solubility and in vivo delivery of plant-derived phenolic materials.

2. Description of the Prior Art

There is a continued need for improved compositions for improving the stability, solubility and in vivo delivery of plant-derived phenolic materials.

Certain naturally-occurring phenolic compounds, such as plant-derived stilbenes, flavones, anthocyanidins, anythocyanins, and bioflavonoids, exert important and potentially beneficial biological activities in living systems. However, many of these compounds are difficult to deliver to living organisms and target tissues in a controlled manner, suffer from poor solubility in aqueous systems, and are subject to oxidation and both thermal-, chemical- and light-induced degradation. A number of these compounds also have very high melting points, which suggests, at least empirically, that they would not be particularly good candidates for incorporation into relatively low-melting point materials that might otherwise act as pharmaceutical carriers for improved delivery.

Hot-melt extrusion (HME) has been used in the production of many different materials, devices, dosage forms and systems. Compounds that otherwise suffer from poor bioavailability when administered to humans and animals due to low aqueous solubility have been processed into solid dispersions, including amorphous solid dispersions, using appropriate excipients and HME, thereby producing materials with improved solubility and bioavailability characteristics.

HME technologies may offer advantages over traditional methods for producing solid dispersions, such as solvent evaporation techniques. These include shorter processing times, reduced environmental pollution and recycling costs due to the elimination of solvents, versatile product forms, and increased efficiency of drug delivery.

Notably, many of the excipients used in HME are potentially thermally unstable, thereby limiting HME processing temperatures based upon excipient stability. Accordingly, most HME processing is conducted at or near the melting point or glass transition temperature of the primary excipient or excipients used in HME formulations. Further, many of the accessory excipients, as well as the biologically active substance of interest present in the formulation produced using HME, such as certain drugs and plant-derived phenolic materials, are unstable when processed at high temperatures. Evidently, the high degree of crystallinity and high melting points of many plant-derived phenolic materials, such as trans-reservation and quercetin (mp 256 and 315° C., respectively), suggest that they would be poor candidates for HME processing, since it is anticipated that these high melting point compounds would require HME processing at temperatures well above those commonly employed for HME, and well above the maximum tolerated temperature of many commonly used excipients.

There remains a need, therefore, for improved compositions for facilitating stability, solubility, and in vivo delivery of plant-derived phenolic materials.

### SUMMARY

The present invention provides a variety of hot melt extruded plant-derived phenolic material compositions, these compositions exhibiting the useful features described herein.

In accordance with an embodiment, the invention provides a hot-melt extruded composition that may include about 20-80% wt. of a plant-derived phenolic material; about 20-85% wt. of one or more edible or bioerodible excipients; about 0-40% wt. of a surface active material; about 0-40% wt. of an oral absorption enhancer; and about 0-10% wt. of one or more pharmaceutical or food grade additives. The composition was hot-melt extruded at a temperature substantially below the melting point of the plant-derived phenolic material to produce a hot-melt extruded composition wherein substantial degradation of the plant-derived phenolic material has not occurred.

It is an object of the present invention to provide a hot melt extruded composition containing a plant-derived phenolic material, and methods of producing and using the same. Further objects, advantages and features of the present invention will be apparent from the detailed description herein.

These and other objects, features and advantages of the present invention will become apparent in light of the following detailed description of preferred embodiments thereof, as illustrated in the accompanying drawings.

### BRIEF DESCRIPTION OF THE FIGURES

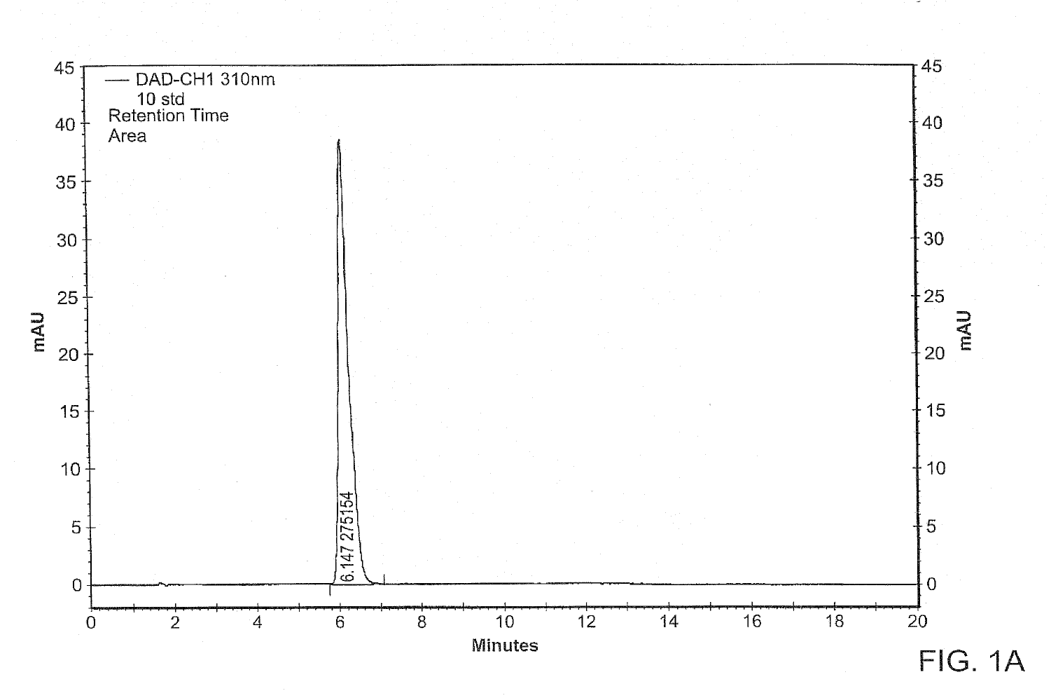

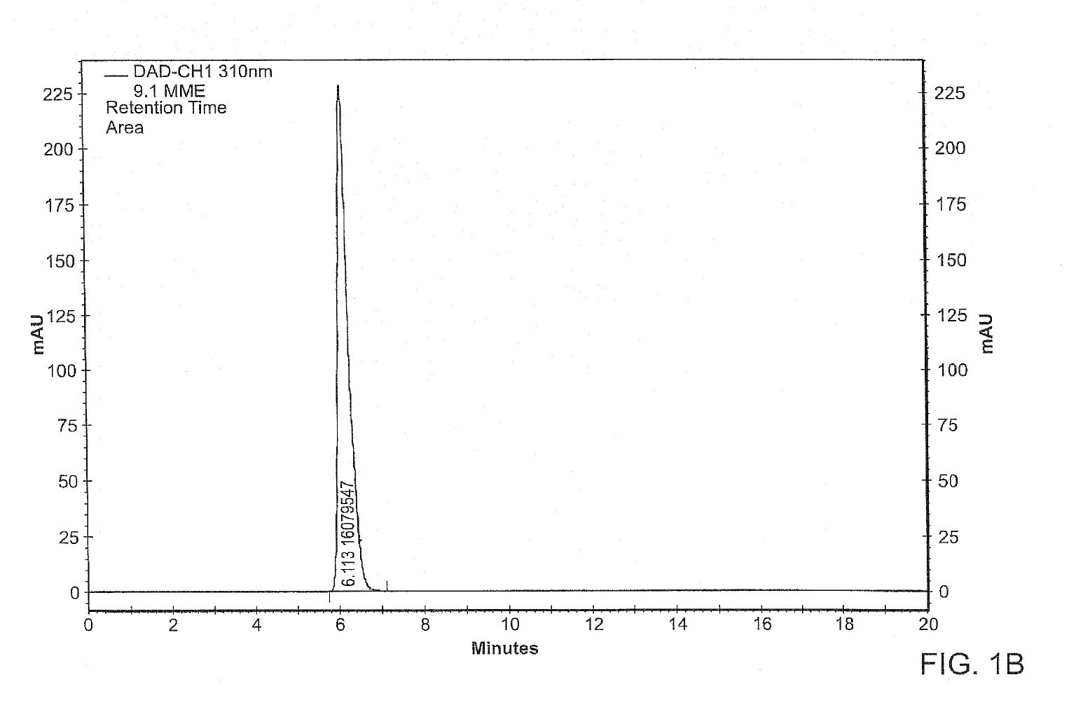

The following description may be further understood with reference to the accompanying Figures in which FIGS. 1A and 1B are HPLC chromatograms of a control, unextruded resveratrol-polymer mixture (FIG. 1A), and the same mixture after hot melt extrusion (FIG. 1B).

### DESCRIPTION OF THE PREFERRED EMBODIMENTS

Applicants have discovered that amorphous solid dispersions of plant-derived phenolic materials dispersed in edible and biocompatible polymers may be produced by hot-melt extrusion at temperatures substantially below the melting point of the plant-derived phenolic material. In particular, it has been discovered that such high-melting, crystalline plant-derived phenolic materials may be incorporated into solid dispersion in an amorphous state when processed using HME at temperatures well below, even less than half, of the melting point of the plant-derived phenolic material. Such dispersions may be produced by HME wherein the plant-derived phenolic material remains substantially stable.

Further still, the improved intrinsic solubility and the controlled rate and extent of release of plant-derived phenolic materials formulated in such HME-produced materials has not been revealed. These plant-derived phenolic materials may, in addition to exerting useful biological effects when released from HME-prepared compositions, also serve as plasticizing, chelating and antioxidant agents when incorporated into HME-produced compositions, thereby serving as useful processing aids and excipients in their own right.

Since hot-melt extrusion may possess many processing advantages useful for the preparation of plant-derived phenolic material-containing compositions, including pharmaceuticals, nutraceuticals, devices, supplements, and dosage forms with useful physical characteristics, a need remains for the development of hot-melt extruded composition containing biologically active plant-derived phenolic materials.

The composition in accordance with certain embodiments, comprises at least one edible, water soluble or water swellable polymer, preferably a polyvinylpyrrolidone polymer, a polyvinylpyrrolidone(copovidone)copolymer, and a methacrylate polymer and copolymer, and a surface active material. The composition may also contain an absorption enhancer, antioxidants, preservatives, flavors and colorants. The composition may contain a conventional plasticizer, or a material which is generally recognized in the art as a plasticizer for hot melt extruded materials. Alternatively, the plant-derived phenolic material may act as a plasticizer or as an antioxidant and additionally, may also act as a colorant and coloring agent. The composition can be milled, ground, comminuted, pulverized, sized, shaped, and otherwise processed and formulated to provide a pharmaceutical dosage form for the controlled delivery of the plant-derived phenolic material to a human or mammal orally for systemic administration, and topically, and to the oral, buccal, rectal, vaginal and otic cavities, and ophthalmically, and as an injectable, and as an implantable, erodible formulation, and as a dissolving implant and particulate.

For purposes of promoting an understanding of the principles of the invention, reference will now be made to particular embodiments of the invention and specific language will be used to describe the same. It will nevertheless be understood that no limitation of the scope of the invention is thereby intended, such alterations and further modifications in the invention, and such further applications of the principles of the invention as described herein being contemplated as would normally occur to one skilled in the art to which the invention pertains.

The invention relates generally to dispersions of plant-derived phenolic compounds produced by hot melt extrusion (HME), wherein said phenolic materials remain substantially stable, and are substantially amorphous, after HME. The composition comprises at least one edible, water soluble or water swellable polymer, preferably a polyvinylpyrrolidone polymer, a polyvinylpyrrolidone(copovidone)copolymer, and a methacrylate polymer and copolymer, and a surface active material. The hot-melt extruded composition is used for in vivo delivery of plant-derived phenolic materials.

The present invention provides compositions comprising amorphous, solid dispersions of plant-derived phenolic materials dispersed in mixtures of pharmaceutical and food grade polymers, excipients and additives, prepared by hot melt extrusion at temperatures below the melting point of the plant-derived phenolic materials. The plant-derived phenolic materials are substantially amorphous, and are substantially stable, without undergoing substantial oxidation, quinone formation, polymerization, phenol coupling, and isomerization.

The compositions may be readily incorporated into foods and pharmaceutical dosage forms for the controlled delivery of plant-derived phenolic materials to a human or a mammal. This invention may also be extended to controlled plant-derived phenolic material delivery in topical, skin care products, vaginal, cranial, abdominal, otic, uterine, nasal, sinus, rectal, buccal, oral, ophthalmic, veterinary and wound care applications, and to adhesive applications, such as for use as a denture adhesive. The present hot-melt extrusion process generally provides shorter and more efficient processing times to a final product, environmental advantages due to elimination of solvents in processing, the production of compositions containing amorphous plant-derived phenolic materials when processed by HME at temperatures below the melting point of the plant-derived phenolic material, substantial plant-derived phenolic material stability, and increased efficiency of drug delivery to a human or mammal.

In one embodiment of the present invention, the intrinsic and equilibrium solubilities of the plant-derived phenolic material dispersed in the HME composition are substantially different from those of the plant-derived phenolic material itself, and from physical mixtures of the plant-derived phenolic material and the corresponding excipients contained in the composition that were not hot melt extruded.

In another embodiment, the rate and extent of the dissolution and release of the plant-derived phenolic material from said composition into an aqueous medium are substantially greater than the rate and extent of the dissolution and release of the plant-derived phenolic material into an aqueous medium, as well as those of the plant-derived phenolic material when dispersed in the unextruded, neat physical mixture of phenolic material and excipients.

In still another embodiment, the rate and extent of the dissolution and release of the plant-derived phenolic material from the composition into an aqueous medium are substantially less than the rate and extent of the dissolution and release of the plant-derived phenolic material into an aqueous medium, as well as those of the plant-derived phenolic material when dispersed in the unextruded, neat physical mixture of phenolic material and excipients.

In one aspect of the invention, the composition is prepared by first mixing all of the individual components in an appropriate mixer, such as a blender, a shaker, a V-blender, and a mill, feeding the mixed material into a hot melt extruder at a controlled rate and at controlled temperature, cooling the extruded material in air, or by a stream of gas, or in a pool of liquid, or on a surface or a moving belt, and recovering the cooled, hot melt extruded material. The HME material may then be used as is, or adapted into a formulation for the controlled delivery of the plant-derived phenolic material to a human or mammal.

In yet another embodiment, the composition is prepared by first mixing one or more of the individual components in an appropriate mixer, such as a blender, a shaker, a V-blender, and a mill, adding one or more of the individual components into a hot melt extruder during the extrusion process at a controlled rate and at controlled temperature, cooling the extruded material in air, or by a stream of gas, or in a pool of liquid, or on a surface or a moving belt, recovering the cooled, hot melt extruded material. The HME material may then be used as is, or adapted into a formulation for the controlled delivery of the plant-derived phenolic material to a human or mammal.

In still another embodiment, the composition is prepared by first mixing one or more of the individual components in an appropriate mixer, such as a blender, a shaker, a V-blender, and a mill, feeding the mixed material into a heated screw hot melt extruder at a controlled rate and at controlled temperature, cooling the extruded material in air, or by a stream of gas, or in a pool of liquid, or on a surface or a moving belt, recovering the cooled, hot melt extruded material, grinding or milling the extruded material into a form that may be fed into a hot-melt extruder, next mixing one or more of the remaining components with the previously extruded material in an appropriate mixer, such as a blender, a shaker, a V-blender, and a mill, and then feeding the mixed material into a hot melt extruder at a controlled rate and at controlled temperature, cooling the extruded material in air, or by a stream of gas, or in a pool of liquid, or on a surface or a moving belt, and recovering the cooled, hot melt extruded material. The HME material may then be used as is, or adapted into a formulation for the controlled delivery of the plant-derived phenolic material to a human or mammal.

In accordance with an embodiment, therefore, the hot-melt extruded composition may include about 20-80% wt. of a plant-derived phenolic material; about 20-85% wt. of one or more edible or bioerodible excipients; about 0-40% wt. of a surface active material; about 0-40% wt. of an oral absorption enhancer; and about 0-10% wt. of one or more pharmaceutical or food grade additives. The composition was hot-melt extruded at a temperature substantially below the melting point of the plant-derived phenolic material to produce a hot-melt extruded composition wherein substantial degradation of the plant-derived phenolic material has not occurred

This versatile composition may be employed for the delivery of a plant-derived phenolic material to a human or mammal as is, or be further incorporated into any number of dosage forms for the same use. Such dosage forms might comprise a bulk powder, a divided powder, a particulate, sprinkles for use with or incorporation into foods, a top dressing for veterinary feed, a tablet triturate, a compressed tablet, a capsule, a liquid capsule, an orally disintegrating tablet, a sublingual tablet, an electuary, a chewing gum, a confection, a semi-solid, a paste, a buccal formulation, a lozenge, a troche, a fast-dissolving film, a suspension, a dispersion, a depot injection, an intra-articular injection, an implant, such as a bioerodible implant, pellet or microsphere, and combinations thereof.

Generally, the hot-melt extruded composition includes a number of different materials and/or additives. These include a plant-derived phenolic material such as a stilbene, a stilbenoid, a flavone, an isoflavone, a flavonoid, a flavonol, a flavanone, a flavan-3-ol, a catechin, an epicatechin, an epigallocatechin, an anthocyanin, an anthocyanidin, a protocyanin, a proanthocyanidin, a condensed tarmin, resveratrol, ellagic acid, punicalagin, gallic acid, piceid, piceatannin, quercetin, rutin, hesperidin, hesperetin, epigallocatechin gallate, aurantinidin, cyanidin, delphinidin, europinidin, luteolinidin, pelargonidin, malvidin, peonidin, petunidin, rosinidin, cyanidin-3-glucoside, cyanidin monoglucuronide, malvidin 3-glucoside, cyanidin 3-glucoside, pelargonidin 3-glucoside, pelargonidin 3-rutinoside and pelargonidin 3-acetylglucoside, cyanidin 3-malonyldiglucoside, pelargonidin 3-malylglucoside, a pelargonidin bioside, a pelargonidin 3-bioside acylated with acetic acid, peonidin 3-O-galactoside, and their glycosides, glucosides, galactosides, arabinosides, and their sulfate esters, and their phosphate esters, and their glucuronides, and pharmaceutically acceptable and food grade salts thereof, and combinations thereof in the range of 20-80% wt.

One or more edible or bioerodible excipients is selected from the group comprising a wax, a fatty acid, such as palmitic acid, stearic acid, and citric acid, a fatty alcohol, such as cetyl alcohol, stearyl alcohol, and cholesterol, a polyvinylpyrrolidone polymer, a polyvinylpyrrolidone(copovidone)copolymer, a polyvinyl alcohol, a cellulose derivative, hydroxypropylcellulose, hydroxypropylmethylcellulose, methylcellulose, ethylcellulose, cellulose acetate, polyethylene oxide, a polyester, such as a polylactide polymer, a polyglycolide polymer, a hydroxybutyrate polymer, and a polylactide-polyglycolide copolymer, a methacrylate polymer, a methacrylate copolymer, an aminoalkyl methacrylate copolymer, polycarbophil, carbomer, one or more acrylic polymers, one or more polyacrylic acids, copolymers of these polymers, such as Soluplus®, pullulan, and combinations thereof in the range of 20-85% wt.

A surface active material such as a polyalkylene glycol polymer, a polyethylene-polypropylene glycol polymer(poloxamer), a polyethylene glycol polymer (PEG), a poly(ethylene oxide polymer (PEO), an alkyl ether, a phospholipid, a sterol, cholesterol, a cholesterol ester, an alkyl sulfonate, and combinations thereof in the range of 0-40% wt. An oral absorption enhancer such as fatty acids, glycerol and PEG esters of fatty acids, phospholipids, a polyethylene glycol ether, and combinations thereof in the range of 0-40% wt. The last material is one or more pharmaceutical or food grade additives such as antioxidant, a coloring agent, a flavoring agent, a taste masking agent, a plasticizer, and combinations thereof in the range of 0-10% wt. of The composition is typically hot-melt extruded at a temperature substantially below the melting point of the plant-derived phenolic material to produce a hot-melt extruded composition such that substantial degradation of the plant-derived phenolic material has not occurred.

The plant-derived phenolic material may be present in a form which is substantially amorphous, microcrystalline, nanocrystalline or a combination of any of the forms. Additionally, the plant-derived phenolic material in the composition may act as a plasticizer.

The composition may be prepared in a variety of methods. Below are just a few examples. All of the individual components are mixed in an appropriate mixer, such as a blender, a shaker, a V-blender, and a mill and then fed into a hot melt extruder at a controlled rate and at controlled temperature. The extruded material is then cooled in air, by a stream of gas, in a pool of liquid, or on a surface or a moving belt. The cooled, hot melt extruded material is recovered and the extruded material is adapted for the controlled delivery of the plant-derived phenolic material to a human or mammal.

Alternatively, the composition is prepared by mixing one or more of the individual components in an appropriate mixer, such as a blender, a shaker, a V-blender, and a mill and then adding one or more of the individual components into a hot melt extruder during the extrusion process at a controlled rate and at controlled temperature. Cooling extruded material in air, by a stream of gas, in a pool of liquid, or on a surface or a moving belt and then recovering the cooled, hot melt extruded material. The extruded material is then adapted for the controlled delivery of the plant-derived phenolic material to a human or mammal.

Still another alternative is to form the composition by first mixing one or more of the individual components in an appropriate mixer, such as a blender, a shaker, a V-blender, and a mill. Then the mixed material is fed into a heated screw hot melt extruder at a controlled rate and at controlled temperature. The extruded material is cooled in air, by a stream of gas, in a pool of liquid, on a surface or on a moving belt wherein the cooled, hot melt extruded material is recovered. The extruded material is ground or milled into a form that may be fed into a hot-melt extruder. Next, one or more of the remaining components is mixed with the previously extruded material in an appropriate mixer, such as a blender, a shaker, a V-blender, and a mill. The mixed material is fed into a hot melt extruder at a controlled rate and at controlled temperature. The extruded material is cooled in air, by a stream of gas, in a pool of liquid, on a surface or a moving belt and the cooled, hot melt extruded material is recovered. The extruded material is then adapted for the controlled delivery of the plant-derived phenolic material to a human or mammal.

With these type of preparations, the hot-melt extruded composition may be extruded as a mass, a film, a sheet, a pellet, a rod, a stick, a particle, a powder, a strand, a disc, an aggregate, and/or combinations thereof.

The compositions may also be adapted into a pharmaceutical or food formulation for the controlled delivery of a plant-derived phenolic material to a human or mammal orally for systemic administration, and topically, and to the oral, buccal, rectal, vaginal and otic cavities, and ophthalmically, and as an erodible implant, and as an injectable particulate. The composition used in the a bulk powder, a divided powder, a particulate, a top dressing for feed, a molded tablet, a tablet triturate, a compressed tablet, a capsule, a liquid capsule, an orally disintegrating tablet, a sublingual tablet, a fast dissolving tablet, an electuary, a chewing gum, a confection, a semi-solid, a paste, a buccal formulation, an adhesive buccal formulation, a lozenge, a troche, an adhesive film, a fast-dissolving film, a suspension, a dispersion, a depot injection, an intra-articular injection, an implant, a bioerodable implant or particle, a dissolvable implant or particle, and combinations thereof.

The useful, versatile invention encompasses a wide variety of compositions and methods of their production and adaptation into formulations and dosage forms. Some exemplary compositions are set forth in the examples. While these are described for purposes of example, it is understood that these examples are not intended to be limiting in any way. Accordingly, the invention will be further described with reference to the following specific examples, which illustrate, but in no way limit, the invention.

### EXAMPLE ONE

Resveratrol 99% 6.9 g, Kollidon VA64 142.56 g, and Lutrol F 68 15.0 g, are mixed intimately in the dry state in a blender.

The material is then introduced into a 16 mm twin-screw hot melt extruder using a Thayer feeder, with a feed rate from about 10 g to about 1000 g per minute. Screw speed is from about 60 to about 100 rpm, head pressure from about 4 to about 20 bar, and torque from about 3 to about 22 Nm.

The extruder is equipped with 6 successive temperature zones, set at 130° C., 130° C., 140° C., 150° C., 150° C., and 140° C., respectively. The material is extruded over a period from about 30 seconds to about 10 min in length, and the resulting extrudate is collected and cooled in a bag as brittle sticks, yielding over 150 g of material. HPLC and DSC analysis reveal that the material comprises approximately 4.6% w/w amorphous resveratrol homogeneously dispersed in the polymer matrix.

This material is comminuted in a twin blade rotary mill, sieved to a standard size fraction of 250-425 μm, and 200 mg is then loaded into #0 capsules. An equivalent amount of a physical mixture of the ingredients in the extruded composition, containing an identical w/w percentage of resveratrol, is similarly loaded into capsules. The capsules are loaded into sinkers and placed in a USP basket dissolution apparatus (500 mL 0.1N HCl/0.3% sodium dodecyl sulfate, speed 75 rpm, temp. 37° C.), and the rate and extent of resveratrol dissolution and release are monitored at regular intervals over 90 min by HPLC. The rate and extent of resveratrol dissolution and release is significantly greater from the extruded material than from the physical mixture control.

FIG. 1A shows HPLC chromatograms of a control, unextruded resveratrol-polymer mixture. FIG. 1B shows HPLC chromatograms of the same mixture after hot melt extrusion. As shown in FIG. 1B, HPLC analysis reveals that resveratrol remains chemically intact after hot melt extrusion.

### EXAMPLE TWO

Kollidon VA64 134.97 g, and Lutrol F 68 15.0 g, are mixed intimately in the dry state in a blender. The mixture is then introduced into a 16 mm twin-screw hot melt extruder using a Thayer feeder. Screw speed is from about 60 to about 100 rpm, head pressure from about 4 to about 16 bar, and torque from about 5 to about 16 Nm.

The extruder is equipped with 6 successive temperature zones, set at 130° C., 130° C., 140° C., 150° C., 150° C., and 140° C., respectively. The material is extruded over a period from about 30 seconds to about 6 min in length, and the resulting extrudate is collected and cooled on an inert metal pan.

This material was next comminuted in a twin blade mill, and mixed intimately with resveratrol 99% 15.1 g, in a blender.

The material is again introduced into a 16 mm twin-screw hot melt extruder using a Thayer feeder. The extruder is equipped with 6 successive temperature zones, set at 130° C., 130° C., 140° C., 150° C., 150° C., and 140° C., respectively, as described in the previous example. The material is extruded over a period from about 30 seconds to about 8 min in length, and the resulting extrudate is cooled on a conveyor as brittle sticks, yielding over 150 g of material comprising approximately 9.0% w/w amorphous resveratrol homogeneously dispersed in the polymer matrix.

### EXAMPLE THREE

Quercetin 99% 7.6 g, Eudragit EPO 142.56 g, and PEG 4000 14.85 g, are mixed intimately in the dry state in a blender.

The material is then introduced into a 16 mm twin-screw hot melt extruder using a Thayer feeder, with a feed rate from about 20 g to about 1000 g per minute. Screw speed is from about 60 to about 100 rpm, head pressure from about 2 to about 14 bar, and torque from about 3 to about 17 Nm.

The extruder is equipped with 6 successive temperature zones, set at 120° C., 120° C., 140° C., 140° C., 140° C., and 135° C., respectively. The material is extruded over a period from about 30 seconds to about 6 min in length, and the resulting extrudate is collected and cooled in a bag as pellets, yielding approximately 140 g of material comprising approximately 4.4% w/w amorphous quercetin homogeneously dispersed in the polymer matrix.

### EXAMPLE FOUR

Quercetin 99% 7.59 g, Kollidon VA64 142.56 g, and Lutrol F 68 14.85 g, are mixed intimately in the dry state in a blender.

The material is then introduced into a 16 mm twin-screw hot melt extruder using a Thayer feeder, with a feed rate from about 10 g to about 1000 g per minute. Screw speed is from about 60 to about 100 rpm, head pressure from about 1 to about 12 bar, and torque from about 2 to about 17 Nm.

The extruder is equipped with 6 successive temperature zones, set at 120° C., 120° C., 140° C., 140° C., 140° C., and 135° C., respectively. The material is extruded over a period from about 30 sec to about 6 min in length, and the resulting extrudate is collected and cooled in a bag as pellets, yielding approximately 143 g of material comprising approximately 4.5% w/w amorphous quercetin homogeneously dispersed in the polymer matrix.

### EXAMPLE FIVE

Resveratrol 99% powder (2 to 10% w/w) is sifted together with PL38 poly-L-lactide bioerodible polymer powder, and then fed manually into a Leistritz 16 mm twin screw extruder at a rate of approximately 5 g/min.

The material is then extruded past 6 successive temperature zones, with set temperatures of 150° C., 175° C., 210° C., 210° C., 215° C. and 210° C., respectively, with a screw speed of 75 rpm, at an average torque of 40 nM, thereby producing a solid dispersion comprising amorphous, chemically-unchanged resveratrol dispersed in PL38 bioerodible polymer, as a strand, a thread, a fiber, a rod, or a stick.

The extruded material is then cooled, and collected as a strand, a thread, a fiber, a rod, or a stick, which may be used as a delivery system as extruded. Alternatively, the extruded material may then be cut into appropriate lengths for further incorporation into a pharmaceutical formulation, such as an implantable, bioerodible delivery system. Alternatively, the material may be pelletized through the use of an in-line or free-standing pellet feed roller and cutter, and the resulting pellets then used as injectable or implantable bioerodible delivery systems for the controlled systemic delivery of polyphenolic compounds. Such injectable or implantable systems might be injected or implanted intramuscularly, subdermally, subcutaneously, intralesionally, or by another acceptable route of administration.

Those skilled in the art will appreciate that numerous modifications and variations may be made to the above disclosed embodiments without departing from the spirit and scope of the invention.

What is claimed is:

1-17. (canceled)

18. A method of preparing a hot-melt extruded composition, said method comprising the steps of:
mixing in a mixer at least about 20-80% wt. of a plant-derived phenolic material, and about 20-85% wt. of one or more edible or bioerodible excipients, and up to 80% wt of any of a surface active material, an oral absorption enhancer, and/or one or more pharmaceutical or food grade additives to provide a mixed material;
feeding the mixed material into a hot melt extruder at a controlled rate and at controlled temperature to provide an extruded material;
cooling the extruded material in air, or by a stream of gas, or in a pool of liquid, or on a surface or a moving belt;
recovering the cooled extruded material.
adapting the cooled extruded material for the controlled delivery of the plant-derived phenolic material to a human or mammal.

19. The method of claim 18, wherein the plant-derived phenolic material is present in a substantially amorphous form.

20. The method of claim 18, wherein the plant-derived phenolic material is present in a substantially microcrystalline form.

21. The method as claimed in claim 18, wherein the plant-derived phenolic material is present in a substantially nanocrystalline form.

22. The method as claimed in claim 18, wherein the plant -derived phenolic material is present in a combination of amorphous, nanocrystalline and microcrystalline forms.

23. The method as claimed in claim 18, wherein the plant-derived phenolic material is selected from the group comprising a stilbene, a stilbenoid, a flavone, an isoflavone, a flavonoid, a flavonol, a flavanone, a flavan-3-ol, a catechin, an epicatechin, an epigallocatechin, an anthocyanin, an anthocyanidin, a protocyanin, a proanthocyanidin, a condensed tannin, resveratrol, ellagic acid, punicalagin, gallic acid, piceid, piceatannin, quercetin, rutin, hesperidin, hesperetin, epigallocatechin gallate, aurantinidin, cyanidin, delphinidin, europinidin, luteolinidin, pelargonidin, malvidin, peonidin, petunidin, rosinidin, cyanidin-3-glucoside, cyanidin monoglucuronide, malvidin 3-glucoside, cyanidin 3-glucoside, pelargonidin 3-glucoside, pelargonidin 3-rutinoside and pelargonidin 3-acetylglucoside, cyanidin 3-malonyldiglucoside, pelargonidin 3-malylglucoside, a pelargonidin bioside, a pelargonidin 3-bioside acylated with acetic acid, peonidin 3-O-galactoside, and their glycosides, glucosides, galactosides, arabinosides, and their sulfate esters, and their phosphate esters, and their glucuronides, and pharmaceutically acceptable and food grade salts thereof, and combinations thereof.

24. The method as claimed in claim 18, wherein the edible or erodible excipient is selected from the group comprising a wax, a fatty acid, such as palmitic acid, stearic acid, and citric acid, a fatty alcohol, such as cetyl alcohol, stearyl alcohol, and cholesterol, a polyvinylpyrrolidone polymer, a polyvinylpyrrolidone(copovidone)copolymer, a polyvinyl alcohol, a cellulose derivative, hydroxypropylcellulose, hydroxypropylmethylcellulose, methylcellulose, ethylcellulose, cellulose acetate, polyethylene oxide, a polyester, such as a polylactide polymer, a polyglycolide polymer, a hydroxybutyrate polymer, and a polylactide-polyglycolide copolymer, a methacrylate polymer, a methacrylate copolymer, an aminoalkyl methacrylate copolymer, polycarbophil, carbomer, one or more acrylic polymers, one or more polyacrylic acids, copolymers of these polymers, such as Soluplus®, pullulan, and combinations thereof.

25. The method as claimed in claim 18, wherein said surface active material is selected from the group comprising a polyalkylene glycol polymer, a polyethylene-polypropylene glycol polymer(poloxamer), a polyethylene glycol polymer (PEG), a poly(ethylene oxide polymer (PEO), an alkyl ether, a phospholipid, a sterol, cholesterol, a cholesterol ester, an alkyl sulfonate, and combinations thereof.

26. The method as claimed in claim 18, wherein the oral absorption enhancer is selected from the group comprising fatty acids, glycerol and PEG esters of fatty acids, phospholipids, a polyethylene glycol ether, and combinations thereof.

27. The method as claimed in claim 18, wherein the pharmaceutical or food grade additive is an antioxidant, a coloring agent, a flavoring agent, a taste masking agent, a plasticizer, or combinations thereof.

28. The method as claimed in claim 18, wherein the plant-derived phenolic material acts as a plasticizer.

29. A method of preparing a hot-melt extruded composition, said method comprising the steps of:
mixing in a mixer one or more of at least about 20-80% wt. of a plant-derived phenolic material, and about 20-85% wt. of one or more edible or bioerodible excipients, and up to 80% wt of any of a surface active material or an oral absorption enhancer or one or more pharmaceutical or food grade additives to provide a mixed material;
feeding the mixed material into a heated screw hot melt extruder at a controlled rate and at controlled temperature;
cooling the extruded;
recovering the cooled, hot melt extruded material;
grinding or milling the extruded material into a form that may be fed into a hot-melt extruder;
mixing one or more of the remaining components with the previously extruded material in a Mixer;
feeding the mixed material into a hot melt extruder at a controlled rate and at controlled temperature;
cooling the extruded material;
recovering the cooled, hot melt extruded material; and
adapting the extruded material for the controlled delivery of the plant-derived phenolic material to a human or mammal.
